(19) United States
(12) Patent Application Publication
(10) Pub. No.: US 2012/0184780 A1
(43) Pub. Date: Jul. 19, 2012

(54) PROCESS FOR THE PREPARATION OF ISOHUMULONE COMPOSITIONS

(75) Inventors: Joshua James Tuinstra, Plainwell, MI (US); Peter Collins Vanalstyne, Paw Paw, MI (US); Brian Patrick Buffin, Kalamazoo, MI (US)

(73) Assignee: KALAMAZOO HOLDINGS, INC., Kalamazoo (US)

(21) Appl. No.: 13/496,940

(22) PCT Filed: Sep. 22, 2010
(86) PCT No.: PCT/US10/02601

§ 371 Date: Mar. 19, 2012

(30) Foreign Application Priority Data
Sep. 22, 2009 (US) 61277228

(51) Int. Cl. C07C 45/80; C07C 49/743

(52) U.S. Cl. 568/341; 568/379

(57) ABSTRACT

A process for preparing isohumulone compositions, which process provides an improvement over the extant art, and yields an isohumulone preparation derived from a hop extract of high yield and purity, which has excellent physical stability, and is essentially free from undesirable lupulones, fatty acids, hop oils and degradation compounds.

### FIELD OF INVENTION

The present invention relates to a process that provides an improvement over the extant art and provides an isohumulone preparation derived from a hop extract of high yield and purity which has excellent physical stability and is essentially free from undesirable lupulones, fatty acids, hop oils and degradation compounds.

### BACKGROUND OF THE INVENTION

The production of beer and other brewed beverages has traditionally involved the addition of hops and hop derivatives thereto. Hop materials impart a distinctive, bitter flavor to brewed beverages. The primary flavoring ingredients in hop cones involve materials known as humulones (alpha acids). In beer brewing, hops are boiled with wort at a pH value around 5.5. Under these conditions, the hop humulones are poorly soluble, but during the process some of the humulones are transformed by isomerization into derivatives known as isohumulones (iso-alpha-acids), which are more soluble in the wort medium. Consequently, to be used efficiently in the production of brewed beverages as flavoring agents, the foregoing humulones must be isomerized to isohumulones.

There are numerous methods by which the isomerization of humulones in hop materials may be achieved. For example, boiling the hop materials in highly alkaline solution will result in isomerization. However, when this process is used, degradation of isohumulones takes place, especially when the pH exceeds 9.5. Degradation occurs due to the fact that isohumulones are particularly unstable in strong alkaline conditions (Verzele, 1991).

U.S. Pat. No. 4,666,731 claims a process that separates the humulones using less than 0.98, and preferably 0.85, equivalents of base relative to humulones, said base selected from sodium and potassium hydroxides, bicarbonates, and carbonates. The alkaline solution is autoclaved at 120° C. for 2.5 hours or exceptionally longer at lower temperatures. Higher temperatures may be used but results in increased degradation of the humulones. This process provides low utilization of humulones, perhaps in part due to low equivalent amount of base used relative to humulones in the initial separation from the extract (see Example 1). It also requires higher temperatures and longer reaction time than the present invention due to the fact that no alkaline earth metal salt capable of catalyzing the isomerization is used.

U.S. Pat. No. 4,758,445 describes a process that consists of mixing hop extract with alkaline aqueous solution in a ratio of 1:2 to 1:50 (pH approximately 9.0) and stirring at elevated temperatures to obtain a two-phase system in which the quasi-aqueous phase containing dissolved humulones is separated. The humulones are precipitated from the aqueous phase by addition of magnesium chloride that forms a chelate with the humulones. This process is repeated multiple times to maximize yield. The alkaline earth metal humulates are collected by filtration, spread as a thin layer on a plate, and isomerized by subjecting them to elevated temperature of around 100° C. and humidity of 90-98% for a period of 5 minutes to 6 hours. The isomerized magnesium isohumulates are diluted in ethanol to approximately a 10% solution, acidified, and subjected to reverse osmosis, providing an isohumulone that is then diluted with ethanol to the desired isohumulone concentration. This process employs solid handling procedures, separation techniques and specific isomerization conditions that are not required in the present invention.

U.S. Pat. No. 3,952,061 claims a process that isomerizes humulone containing material in a medium of water and a water miscible organic solvent, such as methanol or ethanol, with one molar equivalent of a salt such as a magnesium chloride. This process uses water miscible organic solvents and crystallization techniques using isooctane extracts of ethereal solutions to purify isohumulones that are not needed in the present invention.

U.S. Pat. No. 5,015,491 claims a process that isomerizes hop extract, using no solvents or diluents, with a solid alkali or alkaline earth metal compound, preferably 1-4 molar equivalents base to alpha acids, at temperatures preferably in the 120°-140° C. range. This process uses high temperatures with short contact times to produce a highly viscous or brittle solid that can be ground into a fine powder to be used in beer brewing. This process does not employ an isolation technique to purify the isohumulones from the hop extract. The impurities such as fatty acids, lupulones, alkaline earth metal salts and degradation products can produce stability issues in the final beer product in the form or solids, haze and possible undesired flavors that are not encountered when using the present invention.

U.S. Pat. No. 5,370,897 claims a process that combines hop extract with 1.0-4.0 volumes of warm water and isomerizes with 0.1-0.5 molar equivalents of alkaline earth salt per mole of alpha at a temperature greater than 70° C. for 1-3 hours. The alkaline earth resin complex is disassociated by the addition of an acid and the organic layer that forms is used for brewing processes. This process does not employ a purification process to isolate the isohumulones from the rest of the extract. The resulting organic layer includes lupulones, fatty acids and degradation products that are undesirable in the final beer product.

U.S. Pat. No. 5,478,580 claims an aqueous process that combines hop extract, deionized water and a metal salt isomerizing agent in powder form with a weight ratio of 0.2:1 to 0.5:1, isomerizing agent to hop extract. Preferred isomerizing compounds for this process include MgO, Mg(OH)₂, ZnO, Zn(OH)₂, CaO, Ca(OH)₂, and NaOH. The reaction mixture is boiled to complete isomerization and then treated with multiple acid washes at reflux and is partitioned to free the isohumulones from the isomerized metal chelate. This process then uses multiple alkaline pH partitions to isolate the isohumulones from the lupulones and hop oils before being washed with acid again to further purify the isohumulones. The resulting isohumulones in acid form are then diluted with a controlled amount of a monovalent alkaline salt of sodium or potassium, and the resulting solutions can be used in brewing processes. This process isomerizes and acidifies the hop extract prior to isohumulone isolation which will greatly affect the types and amounts of impurities, such as fatty acids and residual alpha acids, which end up in the final extract. These types of impurities are minimized in the present invention, by separating the humulones away from the other extract ingredients prior to isomerization, thereby limiting the types and amounts of impurities that make their way to the beer. The process described in U.S. Pat. No. 5,478,580 also requires multiple washes under various pH conditions at high temperature. These cumbersome processes are minimized or avoided with the present invention which also has the advantage of reducing the amounts of discarded waste streams and salts formed by multiple acid-base dilutions.

U.S. Pat. No. 4,234,516 “Method of Isomerizing Humulone to Isohumulone by Catalytic Acceleration with Metal Salts” from 1980 covers the direct isomerization of humulone or humulone-containing material at elevated temperature and a pH below 9 using a divalent metal ion. Metal catalysts discussed include Zn, Mg, Ca, Ba, Sr, Mn, as well as anions such as acetate, sulfate, and chloride. Their process does not disclose a step wherein the humulone input is separated and purified from the beta acids prior to isomerization. They do report high yields of isohumulones, but they do not specifically discuss purities. Additionally, many of the examples also crystallize the product to purify it, which is not needed in the instant process to obtain high purity isohumulone products.

GB 1,424,785 (publication date=1976) also describes alkaline earth metal compounds as well as zinc oxide and zinc carbonate as isomerization agents. This patent describes a process for isomerizing the alpha acids in a hop extract utilizing divalent metals in a biphasic solution of a water immiscible solvent and a water miscible solvent. They do not isolate alpha acids from the hop extract prior to isomerization. No mention is made of pH control to minimize degradation, nor removal of fatty acids to achieve the product purity necessary to have physical stability of a resulting isohumulone solution in water @ pH 9.0 to 10.0. They claim isolation of the isoalpha acids after isomerization by contacting a water immiscible solvent containing the isoalpha acids with aqueous alkali at pH sufficient to transfer the isoalpha acids into the aqueous phase as their alkali metal salts, but not sufficient to transfer the majority of the beta acids into the aqueous phase. The instant process, on the other hand, removes the majority of the beta acids prior to isomerization, and removes the last traces of beta acids after isomerization via isohexanes/aqueous caustic partitioning. It has been found that removal of beta is critical to physical stability, particularly at low temperatures (˜0° C.).

### OBJECTS OF THE INVENTION

It is an object of the invention to provide an improved process of preparing a composition of purified isohumulones from hop extracts, said isohumulones being essentially free from undesirable lupulones, fatty acids, hop oils and degradation compounds.

It is also an object of the present invention to avoid the disadvantages of prior art methods, such as described hereinabove.

It is another object of the present invention to isolate the humulones from hop extract prior to further processing in a manner that allows the remaining valuable hop chemicals, such as lupulones and hop oils, to be reserved largely unchanged and therefore useful for other purposes.

It is still another object of the present invention to provide a process for the rapid, gentle production of isohumulones using alkaline earth metal salts to accelerate the reaction process.

It is still another object of the present invention to provide an isohumulone product from hop extract in high yields (>90%) and purities (>90%) by isolating the humulones from hop extract, isomerizing said humulones in an accelerated manner by use of zinc or alkaline earth metal salts, and purifying isomerized isohumulones into a product suitable for brewing and other purposes.

### BRIEF SUMMARY OF THE INVENTION

What we therefore believe to be comprised by our invention may be summarized inter alia in the following words:

A method for preparing a purified isohumulone composition, comprising the steps of:
a. dissolving a hop extract comprising humulones in a water-immiscible solvent and mixing in 0.7-1.1 molar equivalents relative to humulone concentration of an aqueous alkaline solution at a temperature of 35-45° C. to form a two phase separation;
b. recovering a humulone-enriched aqueous layer and optionally adjusting the pH to 8.6-9.0 with an aqueous alkaline solution;
c. heating the humulone-enriched aqueous layer to reflux while adding a divalent metal compound as an isomerizing agent;
d. heating the aqueous mixture at reflux under an inert atmosphere until isomerization of humulones to isohumulones is complete;
e. cooling the aqueous mixture to 60-90° C.;
f. adding 0.9-1.2 molar equivalents of an aqueous solution of an acid to isohumulone at 60-90° C. for 0.5-2.0 hours under an inert atmosphere;
g. cooling resulting mixture to 30-45° C. and adding a water-immiscible solvent;
h. stirring the solution, and then separating the organic and aqueous phases;
i. optionally, washing the recovered organic phase with water, by adding water, stirring and separating the phases;
j. optionally, repeating step (i) to remove ionic species;
k. recovering the organic phase and mixing it with 0.25-1 volume of water, warming the mixture to 30-45° C., adjusting the pH to 6.7-7.0 with an alkaline solution, with stirring, and then separating the phases; and
l. recovering, desolventizing and concentrating the aqueous layer containing the purified isohumulones, and adjusting the pH and concentration with an aqueous alkaline solution to a final pH of 9.0-10.0 to a desired concentration while stirring and heating, such a

method wherein the hop extract is from cones of hop plants of the genus Humulus, such a

method wherein the hop cones are extracted by means of solvent extraction or supercritical fluid extraction or any other extraction means known to those skilled in the art, such a

method wherein the aqueous solution of an acid is added in a range of 0.9-1.1 molar equivalents to isohumulone at 60-90° C. for 0.5-2.0 hours under an inert atmosphere, when the isomerization agent is a magnesium compound, such a

method wherein the water-immiscible solvent is a hydrocarbon solvent, such a

method wherein the hydrocarbon solvent is isohexane, such a

method wherein the isohexane is a mixture of saturated hydrocarbons, predominantly of the formula C₆H₁₄, with a boiling point range of about 65 to 71° C., where the major isomers are n-hexane and 2-methylpentane, such a

method wherein the water-immiscible solvent is a mixture of hydrocarbons, such a

method wherein the immiscible solvent is a mixture of hydrocarbons which are predominantly composed of six carbons and varying in their weight ratios, relative to each other, such a

method wherein the volume ratio of hop extract comprising humulones to solvent in step (a) ranges from 0.5-3.0, such a

method wherein said aqueous alkaline solution is selected from one or more of hydroxides of sodium or potassium, such a

method wherein the aqueous alkaline solution is potassium hydroxide, such a

method wherein the divalent metal isomerization catalyst is selected from oxides, hydroxides, sulfates, chlorides, and acetates or other carboxylates, of Mg, Ca, and Ba, and combinations thereof, such a

method wherein the divalent metal isomerization catalyst is selected from zinc oxide, zinc hydroxide, zinc sulfate, zinc chloride, zinc acetate or other carboxylate, and combinations thereof, such a

method wherein the divalent metal isomerization catalyst is MgSO₄ or any of its hydrated forms, such a

method wherein the acid is selected from HCl, H₃PO₄ and H₂SO₄, such a

method wherein the acid is H₂SO₄, such a

method wherein an isohumulone-metal chelate is formed at step (d), and wherein the isohumulone-metal chelate is separated from solution prior to adding the acid, such a

method where in step (1), the isohumulones are desolventized by vacuum drying or any other form of desolventizing known to those skilled in the art to levels of solvent suitable for human consumption, such a

method wherein the recovery yield of starting hop extract humulones to the resulting isohumulones is greater than 70%, such a

method wherein the recovery purity of the resulting isohumulones is greater than about 90%, such a

method wherein the resulting purified isohumulone composition is a suitable additive for bitter flavor in beer brewing processes, such a

a purified isohumulone composition obtained by the method.

### DETAILED DESCRIPTION OF THE INVENTION

This invention relates to a practical and effective process of providing purified isohumulones from hop extract through isolation and isomerization of humulones with minimal steps and handling. The preferred process involves isolation of humulones contained in hop extracts using a hydrocarbon solvent and alkaline aqueous partition, separating the aqueous layer and isomerizing humulones in the aqueous layer to isohumulones using a zinc or an alkaline earth metal salt isomerizing agent. Once isomerization is complete, the isohumulone-divalent metal complex formed is treated with acid and a hydrocarbon solvent to separate the purified isohumulones from the metal ions. The resulting isohumulone hydrocarbon solution is further purified by extraction into an aqueous alkaline solution which can be adjusted to a desired pH and concentration. This process provides an isohumulone product in high yield, purity and stability that is suitable for beer brewing or other uses.

The present invention provides an economical and effective process for isolating humulones from hop extract, isomerizing said humulones to isohumulones, and recovering isohumulones in high yields, high purity and excellent physical stability that is suitable for use in beer brewing and other processes.

### Sources

Humulones, which consist of a number of congeners, including compounds commonly referred to as n-, co- and ad-derivatives are found in the female flower cones, also known as strobiles, of the hop plant (Humulus lupulus). Liquid hop extracts are commercial products which are well known in the art, and are produced by organic solvent extraction as well as supercritical or liquid carbon dioxide extraction of hop cones to remove beer bittering agents such as humulones and lupulones. The present invention shall not be limited to any particular type of hop extract, although extraction by means of low-pressure supercritical carbon dioxide processing is preferred due to high concentration of humulones and lower concentrations of undesirable plant by-products, in particular fatty acids. Low-pressure extracts (<2400 psi) tend to be lower in triglyceride and fatty acid concentrations, generally <1.5% by mass calculated as free fatty acids (FFA), than extracts of higher pressures (3800-4500 psi), generally 2.5-6% FFA (Chrastil, 1982; Ribeiro and Bernardo-Gil, 1995; Garlapati and Madras, 2008). The pH and temperature encountered in the humulone isomerization process hydrolyze any triglycerides present into free fatty acids and glycerol. The free fatty acids are problematic in high concentrations and crash out of solution to form a haze in the final solution.

The solubility behavior of fatty acids in the final product varies based on the number of carbon atoms, pH, temperature, etc. Fatty acids typically contain anywhere from about eight to twenty-two carbon atoms. Examples of these fatty acids include linoleic, palmitic, oleic, linolenic, myristic, stearic, lauric, and the like. As the chain length increases the solubility of the fatty acids in water decreases (Reiger and Rhein, 1997).

### Isolating Humulones

Isolating humulones from hop extract prior to processing allows the remaining valuable hop chemicals, such as lupulones and hop oils, to be reserved for other purposes with minimal modifications of their chemical properties due to the temperature, pH and other processing conditions required in the isomerization process. Isolating humulones from extract prior to processing can be achieved due to the solubility characteristics of humulones compared to the other organic hop constituents, providing material in high yields and purities for isomerization starting material. Isolating humulones from extract in relatively high purities is important to remove a majority of lupulones and fatty acids, in particular fatty acids with greater than or equal to 16 carbons in chain length, that result in solid and haze formation in the final product due to their poor solubility.

To isolate humulones, the hop extract is dissolved in an equal volume of a hydrocarbon solvent such as isohexane. Isohexane is defined as a mixture of saturated hydrocarbons, predominantly of the formula C₆H₁₄, hereafter referred to as isohexane(s). This process can also be done without isohexane, but the use of isohexane helps to create a cleaner partition with higher yields of humulones in the aqueous partition and lower levels of lupulones and fatty acids (see Example 2), which will produce solids and haze formation in the final product if not removed (Foster, 1995). The solution is mixed with a 3% potassium hydroxide (KOH) aqueous solution, using about a 0.9-1.1 (preferably 1.1) molar equivalent of base to humulone, thereby increasing the solubility of the humulones and providing a pH of about 8.6 to 9.0. The mixture is stirred for 10 to 20 minutes at a temperature of about 35 to 45° C. KOH reacts with humulones (alpha acids) to form water soluble potassium salts of humulones that are easily partitioned away from the other constituents of the extract, which remain largely in the isohexane (or organic) layer.

After stirring, the organic phase and aqueous phase are separated. The humulone-enriched aqueous phase, which contains 70 to >97% of the starting humulones, depending on the molar equivalents of KOH used (see Example 2), is collected and the pH is adjusted to 8.9 to 9.2 by the addition of 10% potassium hydroxide in preparation for isomerization. It is important that the pH not exceed 9.5. High pH increases the rate of formation of degradation compounds, such as allo-isohumulones and humulinic acid, during isomerization, which lowers the purity of the final product and in the extreme causes a haze in the final product (Goldstein et al., 1988). The variables described in this step can be optimized based on starting extract to contain a humulone-enriched aqueous partition with low levels of lupulones (preferably <0.5%) and fatty acids (preferably <0.1%) with optimal yield of humulones by those skilled in the art.

### Isomerizing Humulones

The humulone-enriched aqueous solution is mixed and heated to reflux under an atmosphere of nitrogen or other inert gas. Reflux temperatures are needed to ensure complete isomerization in a relatively short amount of time. Once the solution has reached reflux, 0.1-1.0 molar equivalent of an aqueous solution (or powder form) of a divalent alkaline earth metal salt, relative to humulones, is added slowly to minimize solid formation. Exemplary alkaline earth metal salts suitable as isomerizing agents include but should not be limited to oxides, hydroxides, sulfates, chlorides, acetate or other carboxylates of Mg and Ca, where MgSO₄ is an excellent catalyst. Although it is not an alkaline earth metal ion, Zn(II), which is used by brewers to control yeast growth in the process of brewing, is also an effective isomerization catalyst, and in the discussion that follows, zinc should also be considered where alkaline earth metals are discussed. The fact that brewers already use zinc in the brewing process is seen as an advantage in using it in the isomerization of hop acids. Examples of Zn compounds include, but should not be limited to, the oxide, hydroxide, sulfate, chloride, and acetate or other carboxylates of Zn(II). The amount of isomerizing zinc or alkaline earth metal salt agent will impact the reaction time and the distribution of cis- and trans-isohumulones in the final product. The ratio of cis- to trans-isohumulones is about 1.4 under isomerization conditions used without addition of the alkaline earth metal salt. In comparison, the ratio of cis- to trans-isohumulones varies from about 2.3 to 4.0 by addition of 0.1 to 1.0 molar equivalents, respectively, of magnesium sulfate, relative to the humulones, using the instant process. An amount of 0.4 molar equivalent of an aqueous solution of MgSO4 relative to humulones provides a quick reaction time, low impact on reaction pH and, as mentioned previously, higher ratios of the more soluble and stable cis-isomers using the minimal amount of metal ions (see Example 3). A similar increase in the ratio of cis- to trans-isohumulones was observed when a zinc isomerization catalyst is used. The ratio of cis-isohumulones to trans-isohumulones in the product was calculated to be 3.5 for the zinc catalyst used in Example 6. The reaction mixture is heated at reflux under an atmosphere of an inert gas such as nitrogen for about 1.25 hours or until isomerization is complete. Reaction completion (>98% humulone isomerized to isohumulone) can be checked by using high pressure liquid chromatography (HPLC), ultraviolet (UV) spectroscopy or any other method known to those skilled in the art. Once the reaction is complete the reaction is cooled to 85° C.

### Removing Metal Ions

The isohumulone-enriched solution contains isohumulone chelates of metal ions that must be separated. Low pH is needed to release zinc and magnesium ions from the hop acid chelate. The metal ions need to be separated from the hop acids and removed, otherwise solids and haze formation in the final product will occur. In order to break the metal chelate that has formed, the reaction mixture is mixed with a solution of about 1.0 molar equivalents (relative to the isohumulones) of 35% sulfuric acid (H₂SO₄) and stirred at 85° C. for approximately 1 hour under an atmosphere of an inert gas. The amount of acid added can be optimized by those skilled in the art to effectively break the zinc or alkaline earth metal-isohumulone chelate based on the isomerizing metal salt agent and acid used. The chelates of zinc require more acid than do the magnesium chelates to effectively break the chelate and recover the isohumulones in good yield and purity (1.2 molar equivalents of sulfuric acid relative to isohumulones compared to 0.9 to 1.1 molar equivalents for magnesium chelates). The mixture is then cooled to 40° C. and an equal volume of isohexane is added. Isohexane is used to separate the acid-form of the isohumulones from the aqueous solution, which contains high magnesium, sulfate, and hydrogen ion concentrations. The amount of isohexane used can be varied, but 0.85 volumes, relative to the volume of the reaction mixture works well. The resulting solution is stirred and then the organic isohexane phase and aqueous phases are separated. The organic phase is recovered and washed by thoroughly mixing with about one third volume of water at 40° C. and separated to ensure thorough washing of the isohexane layer. This wash step can be optionally repeated with another aliquot of water. Reverse osmosis (RO-grade) water can be used to help remove residual ionic species from isohexane layer. The resulting acidic isohumulone concentrate is relatively free of metal salts (see Example 4).

### Purifying Isohumulones

The isohumulone-enriched organic layer can be further purified to remove residual lupulones and fatty acids that have been carried through the process. Lupulones and fatty acids are less soluble than the preferred isohumulones and are therefore removed to avoid the formation of precipitates and haze in the final product. The oxidation of unsaturated fatty acids, especially linoleic acid, can produce undesired flavors (cardboard flavor) due to the formation of (E)-2-nonenal (Vanderhaegen, 2006). To remove residual lupulones and fatty acids, the mixture is stirred at 40° C. and the pH is adjusted to 6.7 to 7.0 with 10% KOH for about 20 minutes and then the phases are separated. Slightly elevated temperatures help prevent the formation of gums during this process step and shorten pH stabilization time. The aqueous layer containing purified isohumulones is recovered, desolventized and concentrated. The purified isohumulone concentrate material (generally >90% purity) is relatively free of lupulones and fatty acids (see Example 5). The concentrate is diluted with water to the desired concentration while stiffing and heating to 40-60° C. Aqueous KOH is used to adjust the solution to a final pH of 9.0 to 10.0. Warming ensures complete dissolution of isohumulones during these steps of the process. The resulting product provides an isohumulone preparation suitable for beer brewing processes with high yield and purity which has excellent physical stability and is moreover described to be essentially free from undesirable lupulones, fatty acids, hop oils and degradation compounds.

A cold-stability test can also be used to predict the physical stability of the final product. The cold-stability test for the isohumulone product consists simply of cooling the final solution to 0° C. for 24 hours and visually inspecting the solution. If the product remains clear after 24 hours at 0° C. it is likely the solution will remain clear and stable for an extended period of time when stored at ambient conditions. However, if the product produces precipitate or haze after 24 hours at 0° C. then it is likely that the final product will not be stable due to various impurities discussed earlier such as fatty acids and lupulones (see Example 5).

### EXAMPLES

### Example 1

Supercritical CO₂ hop extract (50.03 g, extracted at a pressure of about 2200 psi), containing 51.40% humulones, was mixed with 1 volume of isohexane by overhead stirring in a 500 mL round bottom flask (RBF) until the extract dissolved. Aqueous 3% KOH (150.01 g) solution was added to the mixture to provide approximately 1.1 molar equivalents of KOH to humulones. The mixture was stirred for 20 minutes at 40° C., transferred to a 500 mL separatory funnel and allowed to separate for 30 minutes. The lower aqueous phase was collected and analyzed (results in Table 1 “Humulone Isolation” step). The pH of the humulone-enriched aqueous phase was adjusted from 8.6 to 9.0 with an aqueous 10% KOH solution and heated to reflux (˜104° C.) in a 500 mL RBF under a purge of nitrogen. Once the solution reached reflux, 0.4 molar equivalents (relative to humulone) of an aqueous MgSO₄ solution (7.12 g MgSO₄ heptahydrate in 21 mL RO-grade water) was added slowly to the reaction flask. The reaction was stirred for 1.25 hours at reflux and then analyzed by HPLC to show that >99% of the humulones were isomerized to isohumulones (see step “Post-Isomerization” in Table 1). The reaction was cooled to 85° C. and mixed with 20.23 g of 35% H₂SO₄, which is 1.0 molar equivalent H₂50₄ relative to isohumulones. The resulting mixture was stirred for one hour. The solution was cooled to 40° C., mixed with one volume isohexane for 20 minutes and transferred to a 500 mL separatory funnel. The organic phase was recovered, mixed with one-third volume of water at 40° C. and separated to ensure thorough washing of the isohexane layer. Reverse osmosis (RO-grade) water was used to remove residual ionic species from the isohexane layer. The resulting acidic isohumulone isohexane layer was relatively free of metal salts (see step “Acid/Water Wash” in Table 1). An optional second wash can be performed if the metal salt level is too high at this point. The isohexane layer was further purified by mixing it with one third the volume of RO-grade water at 40° C. and adjusting the pH to 7.0 with 10% KOH in a RBF. The solution was transferred to a separatory funnel and allowed to separate. The lower aqueous layer was collected, desolventized by rotary evaporation to remove residual solvents and analyzed (see “Purified Material” step in Table 1).

TABLE 1
Experimental results for Example 1.

| | | | | | Fatty | | % Yield Isohumulone |
|---|---|---|---|---|---|---|---|
| | Mass | Humulone | Lupulone | Isohumulone | Acids | Residual | from humulone |
| Step | (g) | (%) | (%) | (%) | (%) | Mg⁺² (mg/kg) | in extract |
| Starting Extract | 50.03 | 51.40 | 13.80 | 0.00 | 0.88 | 0.00 | 0.00 |
| Humulone Isolation | 177.45 | 14.32 | 0.44 | 0.00 | 0.13 | 0.00 | 0.00 |
| Post-Isomerization | 176.49 | 0.03 | 0.47 | 14.17 | 0.09 | 2870.00 | 97.25 |
| Acid/Water Washed | 50.35 | 0.01 | 0.25 | 49.33 | 0.11 | <10 | 96.58 |
| Purified Material | 37.27 | 0.00 | 0.00 | 64.32 | 0.03 | <10 | 93.21 |

The resulting isohumulone concentrate was diluted with water and adjusted to a pH of 9.2 with 10% KOH to a concentration of 30% isohumulones. The final solution contained isohumulones with HPLC purity of 94.36%, based on peak areas, and yielded 93.21% of the extract's original humulones as isohumulones and was moreover described to be essentially free from undesirable lupulones, residual humulones and fatty acids.

### Example 2

The amount of humulones extracted from the hop extract was dependent on the molar equivalents of KOH added. Isohexane was added to dissolve the extract, assist in partitioning and provide a cleaner cut of aqueous humulone to isomerize with minimal change to the valuable chemicals remaining in the hop extract, such as lupulones and hop oils. The humulones were separated from the hop extract by preferably dissolving the hop extract with one volume of isohexane. The solution was mixed with a 3% KOH aqueous solution at 0.9-1.1 molar equivalent to humulone which provided a pH of approximately 8.2-9.0. The solution was mixed for 10-20 minutes at 35-45° C. After stirring, the organic layer and humulone-enriched aqueous layers were separated. The separation can be optimized to obtain the highest yield of humulones with minimal lupulone and fatty acid (preferably <0.1%) concentrations by anyone skilled in the art based on the extract being used. A series of separations were performed on hop extract obtained by means of low-pressure supercritical carbon dioxide extraction to show yield differences using KOH molar equivalents of 0.9, 1.0, 1.1 (all with isohexane) and 1.1 without isohexane. The results for the humulone-enriched aqueous layers are shown in Table 2.

TABLE 2
Experimental results for Example 2.

| | KOH | | | | Isolated | |
|---|---|---|---|---|---|---|
| | Equivalents | Isohexane/ | Humulone | Lupulone | Yield of | % Fatty |
| ID | (mol) | Solventless | (%) | (%) | Humulone (%) | Acids |
| 1 | 1.1 | isohexane | 14.2 | 0.29 | 98.2 | 0.04 |
| 2 | 1.1 | solventless | 13.5 | 0.61 | 91.7 | 0.19 |
| 3 | 1.0 | isohexane | 14.0 | 0.17 | 87.0 | 0.07 |
| 4 | 0.9 | isohexane | 13.0 | 0.10 | 72.0 | 0.01 |

The separation that produced the highest yield of humulones with minimal lupulones and fatty acids was sample ID #1 which yielded >98% of the humulones from the starting extract. After separation, the humulone-enriched aqueous layer was adjusted to a pH of 8.9-9.2 with 10% KOH in preparation of isomerization. This pH range enhanced the rate of the reaction while remaining below the higher pH settings that promoted humulone degradation.

### Example 3

The amount of isomerizing alkaline earth metal salt agent can impact reaction time and cis/trans levels of isohumulones in the final product. A series of reactions were performed using optimal aqueous humulone-enriched material from Example 2 to show the effects of various molar equivalents of MgSO₄ on the resulting isohumulone product. Results of these experiments are shown in Table 3.

TABLE 3
Experimental results for Example 3

| | | | Result after Isomerization | |
|---|---|---|---|---|
| | Mg Equivalents | Reaction | Residual | Cis/Trans |
| ID | (mol) | Time (hours) | % Humulone | Isomer Ratio |
| 1 | 0.1 | 5.25 | 0.65 | 2.32 |
| 2 | 0.2 | 1.5 | 0.39 | 3.02 |
| 3 | 0.3 | 1 | 0.05 | 3.43 |
| 4 | 0.4 | 0.75 | 0.08 | 3.83 |
| 5 | 0.5 | 0.25 | 0.09 | 3.88 |
| 6 | 1.0 | <0.25 | 0.04 | 3.94 |

It is important to minimize the amount of metal compositions being used so they can be effectively removed later in the process. The preferred amount of MgSO₄ used in this process was, but is not limited to, 0.4 molar equivalents relative to the amount of humulones in the reaction mixture. After reaction completion, the reaction was cooled to 85° C. Reaction completion (>98% humulone isomerized to isohumulone) can be checked by high pressure liquid chromatography (HPLC), ultraviolet (UV) spectroscopy or any other method known to those skilled in the art.

### Example 4

Magnesium ions need to be separated from the hop acids and removed otherwise solids and haze formation in the final product will occur. To break the magnesium isohumulone chelate, an aqueous 35% sulfuric acid (H₂SO₄) solution was added to provide 1.0 molar equivalent (relative to isohumulone), stirred by vigorous overhead stirring mechanism and heated at 85° C. for 1 hour under an atmosphere of nitrogen. After one hour the mixture was cooled to 40° C. and an equal volume of isohexane was added. The solution was stirred for approximately 15 minutes and then allowed to separate. The organic phase was recovered and mixed with one third volume of water at 40° C. for 15 minutes and again separated to ensure a thorough washing of the isohexane layer. Reverse osmosis (RO-grade) water was used for this wash to remove residual ionic species from the isohexane layer. An additional water wash can be performed, if needed, to remove residual ionic species. The resulting acidic isohumulone concentrate was relatively free of metal salts. A series of experiments were preformed to show the effects of various molar equivalents of H₂SO₄ to isohumulone using material made with the most optimal conditions from Example 3.

TABLE 4
Experimental results for Example 4

| | H₂SO₄ Equivalents | Resulting | Residual Mg⁺² | Isohumulone |
|---|---|---|---|---|
| ID | (mol) | pH | ions (mg/kg) | Retained (%) |
| Start | 0.0 | — | 2870.0 | — |
| 1 | 0.5 | 2.98 | 456.0 | 78.9 |
| 2 | 0.7 | 2.45 | 71.0 | 94.2 |
| 3 | 0.9 | 2.16 | <10 | 99.9 |
| 4 | 1.0 | 1.74 | <10 | 100.0 |
| 5 | 1.1 | 1.40 | <10 | 99.9 |

A 1.0 molar equivalent of H₂SO₄ to isohumulone is preferred to ensure complete disassociation of magnesium and isohumulone. The acidic isohumulone concentrates were mixed with one third the volume of water at 40° C. in preparation for the further purification of the isohumulones as described in Example 5.

### Example 5

The acidic form of isohumulone prepared by the optimal process in Example 4 is further purified to remove residual lupulones and fatty acids that have been carried through the process. Lupulones and fatty acids are less soluble than the preferred isohumulones and can therefore be removed to avoid appearing as precipitate and haze in the final product. A majority of the lupulones were removed in the humulone isolation step (Example 2) and the residual lupulones should be easily partitioned away at a pH <9.0. To remove residual fatty acids the mixture was stirred at 40° C. and the pH was adjusted to 6.7 to 7.0 with 10% KOH. The mixture was stirred for 20 minutes and then the phases were allowed to separate in a separatory funnel. The aqueous layer solubilized the isohumulones while leaving the residual lupulones and a majority of the fatty acids in the isohexane layer. The aqueous isohumulone-enriched layer was collected, desolventized and concentrated to remove residual levels of isohexane. A series of experiments were performed to demonstrate various levels of pH and their effectiveness in removing residual lupulones and fatty acids from the isohumulone product (see Table 5).

TABLE 5
Experimental results for Example 5.

| | | | % Yield | | | |
|---|---|---|---|---|---|---|
| | | | Isohumulone | % Fatty acids in | HPLC % | |
| | | Residual | from humulone in | 30% isohumulone | Purity of | |
| ID | pH | % beta | starting extract | product | Isohumulone | Cold Test |
| 1 | 5.50 | <0.05 | 50.27 | 0.00 | 89.33 | Clear |
| 2 | 6.10 | <0.05 | 89.42 | 0.01 | 90.86 | Clear |
| 3 | 6.30 | <0.05 | 92.04 | 0.01 | 90.84 | Clear |
| 4 | 6.50 | <0.05 | 93.61 | 0.02 | 91.91 | Clear |
| 5 | 6.70 | <0.05 | 93.61 | 0.02 | 91.73 | Clear |
| 6 | 7.00 | <0.05 | 96.17 | 0.02 | 91.93 | Clear |
| 7 | 7.30 | <0.05 | 93.96 | 0.05 | 91.76 | Clear |
| 8 | 7.60 | <0.05 | 94.72 | 0.07 | 91.52 | Clear |
| 9 | 8.00 | <0.05 | 90.70 | 0.11 | 90.6 | Slight Haze |
| 10 | 8.50 | <0.05 | 90.12 | 0.24 | 90.11 | Haze |
| 11 | 9.00 | <0.05 | 89.65 | 0.65 | 89.81 | Haze |
| 12 | 9.50 | 0.33 | 89.42 | 1.10 | 88.12 | Haze |

The purified concentrate was desolventized to remove residual solvents, diluted with water to the desired concentration, and the solution pH was adjusted to 9.0 to 10.0 with aqueous KOH to 40°-60° C. Warming ensured complete dissolution of isohumulones. Each of the samples made above were subjected to the cold test by placing approximately 20 mL of final product in a freezer at 0° C. for 24 hours. After 24 hours the samples were visually observed for clarity. The final products with minimal impurities remained clear while final products with more impurities (in particular >0.1% fatty acids) showed a few particulate or many that produced a haze after 24 hours. Results for the cold test are also shown in Table 5. Conditions that produce a haze in the final product using the cold test should be avoided to ensure a high quality, stable product.

### Example 6

A 3-neck 500-mL round bottom flask equipped with a magnetic stir bar was charged with an aqueous solution of humulones (230 g, 14.2% humulones, Example 2 ID No. 1). The pH was adjusted from 8.7 to 9.0 with a small amount of 10% KOH, and the solution was warmed to reflux under a purge of purified nitrogen gas. After solution reflux had been achieved, a solution of Zn(II) ions, which was prepared by dissolving 8.2 g of zinc acetate dihydrate in 50 mL of RO-grade water, was slowly added under a positive flow of nitrogen to provide a 0.4 molar equivalent of zinc ions relative to humulones. The mixture was heated at reflux under nitrogen for 1.7 hours and then cooled to ambient temperature, which resulted in the precipitation of a solid containing a chelate of zinc and isohumulones. After decanting the liquid phase from the solid, 35% sulfuric acid solution (32 g, 1.2 molar equivalents) was added to the solid. Heating to 92° C. with stirring afforded an orange oil in which the zinc-isohumulone chelate had been broken. Isohexane (300 mL) was added to the previously decanted liquid, and to this was added the warm acidic mixture of isohumulones with rapid stirring. The resulting isohumulone-enriched isohexane layer was then separated from the aqueous salt solution. Residual ions were removed from the isohexane layer by washing with RO-grade water (2 x 100 mL). Water (80 mL, RO-grade) was added to the isohexane layer containing the isohumulones and the mixture was warmed to 40° C. Potassium hydroxide solution (10%) was slowly added with stiffing to raise the solution pH from 2.7 to a value of 6.9. The lower aqueous layer, which contained the isohumulones, was separated from the isohexane layer containing residual non-isomerized humulones, lupulones, and fatty acids. Rotary evaporation was used to desolventize and concentrate the aqueous isohumulone solution. The final solution contained 23 g of isohumulones (71% yield from humulones) with an HPLC purity of 93%. The ratio of cis-isohumulones to trans-isohumulones in the product was calculated to be 3.5 based on HPLC peak areas, which is consistent with the ratio observed when alkaline earth metal salts are used as isomerizing agents.

### REFERENCES

1. Chicoye, Etzer; Fly, Walter H.; Goldstein, Henery, U.S. Pat. No. 4,759,941, Published Jul. 26, 1988.
2. Foster, Robert T., U.S. Pat. No. 5,478,580, Published Dec. 26, 1995.
3. Goldstein, Henry; Ting, Patrick L.; Chicove, Etzer; Goetzke, Gary; Cowles, John M., U.S. Pat. No. 4,767,640, Published Aug. 30, 1988.
4. Kliisters, Paul, U.S. Pat. No. 4,758,445, Published Jul. 19, 1988.
5. Koller, Horst; Hard, Alfons E.; Kirchner, Gerhard, U.S. Pat. No. 3,952,061, Published Apr. 20, 1976.
6. Laws, Derek R. J., U.S. Pat. No. 4,780,330, Published Oct. 25, 1988.
7. Paul, Herbert, U.S. Pat. No. 5,155,276, Published Oct. 13, 1992.
8. Smith, Robert J.; Wilson, Richard J. H., U.S. Pat. No. 5,370,897, Published Dec. 6, 1994.
9. Todd, Paul H., U.S. Pat. No. 4,666,731, Published May 19, 1987.

110. Westwood, Keith T.; Crescenzi, Allan, U.S. Pat. No. 5,015,491, Published May 14, 1991.
11. Verzele, M.; Keukeleire, D., Chemistry and Analysis of Hop and Beer Bitter Acids, Elsevier, New York, 1991; pp 141-179.
12. Chrastil, J. J. Phys. Chem., 1982, 86, 3016-3021.
13. Ribeiro, M. A. and Bernardo-Gil, M.G. J. Chem. Eng. Data 1995, 40, 1188-1192.
14. Garlapati, C. and Madras, G. J. Chem. Eng. Data 2008, 53, 2913-2917.
15. Rieger, Martin M. and Rhein, Linda D., Eds., “Surfactants in Cosmetics”, 2^{nd} Edition, Revised and Expanded (Surfactant Science Series, Vol 68); Marcel Dekker: New York, 1997, p. 8.
16. Vanderhaegen, Bart; Neven, Hedwig; Verachtert, Hubert; Derdelinchx, Guy, “The chemistry of beer aging—a critical review”, Food Chemistry, 95, 2006, 357-381.

1. A method for preparing a purified isohumulone composition, comprising the steps of:
a. dissolving a hop extract comprising humulones in a water-immiscible solvent and mixing in 0.7-1.1 molar equivalents relative to humulone concentration of an aqueous alkaline solution at a temperature of 35-45° C. to form a two phase separation;
b. recovering a humulone-enriched aqueous layer and optionally adjusting the pH to 8.6-9.0 with an aqueous alkaline solution;
c. heating the humulone-enriched aqueous layer to reflux while adding a divalent metal compound as an isomerizing agent;
d. heating the aqueous mixture at reflux under an inert atmosphere until isomerization of humulones to isohumulones is complete;
e. cooling the aqueous mixture to 60-90° C.;
f. adding 0.9-1.2 molar equivalents of an aqueous solution of an acid to isohumulone at 60-90° C. for 0.5-2.0 hours under an inert atmosphere;
g. cooling resulting mixture to 30-45° C. and adding a water-immiscible solvent;
h. stirring the solution, and then separating the organic and aqueous phases;
i. optionally, washing the recovered organic phase with water, by adding water, stiffing and separating the phases;
j. optionally, repeating step (i) to remove ionic species;
k. recovering the organic phase and mixing it with 0.25-1 volume of water, warming the mixture to 30-45° C., adjusting the pH to 6.7-7.0 with an alkaline solution, with stirring, and then separating the phases; and
l. recovering, desolventizing and concentrating the aqueous layer containing the purified isohumulones, and adjusting the pH and concentration with an aqueous alkaline solution to a final pH of 9.0-10.0 to a desired concentration while stirring and heating

2. The method of claim 1, wherein the hop extract is from cones of hop plants of the genus Humulus.

3. The method of claim 2, wherein the hop cones are extracted by means of solvent extraction or supercritical fluid extraction or any other extraction means known to those skilled in the art.

4. The method of claim 1, wherein the aqueous solution of an acid is added in a range of 0.9-1.1 molar equivalents to isohumulone at 60-90° C. for 0.5-2.0 hours under an inert atmosphere, when the isomerization agent is a magnesium compound.

5. The method of claim 1, wherein the water-immiscible solvent is a hydrocarbon solvent.

6. The method of claim 5, wherein the hydrocarbon solvent is isohexane.

7. The method of claim 6, wherein the isohexane is a mixture of saturated hydrocarbons, predominantly of the formula C₆H₁₄, with a boiling point range of about 65 to 71° C., where the major isomers are n-hexane and 2-methylpentane.

8. The method of claim 1, wherein the water-immiscible solvent is a mixture of hydrocarbons.

9. The method of claim 8, wherein the water-immiscible solvent is a mixture of hydrocarbons which are predominantly composed of six carbons and varying in their weight ratios, relative to each other.

10. The method of claim 1, wherein the volume ratio of hop extract comprising humulones to solvent in step (a) ranges from 0.5-3.0.

11. The method of claim 1, wherein said aqueous alkaline solution is selected from one or more of hydroxides of sodium or potassium.

12. The method of claim 1, wherein the aqueous alkaline solution is potassium hydroxide.

13. The method of claim 1, wherein the divalent metal isomerization catalyst is selected from oxides, hydroxides, sulfates, chlorides, and acetates or other carboxylates, of Mg, Ca, and Ba, and combinations thereof.

14. The method of claim 1, wherein the divalent metal isomerization catalyst is selected from zinc oxide, zinc hydroxide, zinc sulfate, zinc chloride, zinc acetate or other carboxylate, and combinations thereof.

15. The method of claim 13, wherein the divalent metal isomerization catalyst is MgSO₄ or any of its hydrated forms.

16. The method of claim 1, wherein the acid is selected from HCl, H₃PO₄ and H₂SO₄.

17. The method of claim 16, wherein the acid is H₂SO₄.

18. The method of claim 1, wherein an isohumulone-metal chelate is formed at step (d), and wherein the isohumulone-metal chelate is separated from solution prior to adding the acid.

19. The method of claim 1, where in step (1), the isohumulones are desolventized by vacuum drying or any other form of desolentizing known to those skilled in the art to levels of solvent suitable for human consumption.

20. The method of claim 1, wherein the recovery yield of starting hop extract humulones to the resulting isohumulones is greater than 70%.

21. The method of claim 1, wherein the recovery purity of the resulting isohumulones is greater than about 90%.

22. The method of claim 1, wherein the resulting purified isohumulone composition is a suitable additive for bitter flavor in beer brewing processes.

23. A purified isohumulone composition obtained by the method of claim 1.
